(19) United States
(12) Patent Application Publication
(10) Pub. No.: US 2024/0342459 A1
(43) Pub. Date: Oct. 17, 2024

(54) BLOOD PUMP

(71) Applicants: Abiomed Europe GmbH, Aachen (DE); ABIOMED, Inc., Danvers, MA (US)

(72) Inventors: Thorsten SIESS, Aachen (DE); Frank KIRCHHOFF, Aachen (DE); Dion MRAZ, Danvers, MA (US)

(73) Assignees: Abiomed Europe GmbH, Aachen (DE); ABIOMED, Inc., Danvers, MA (US)

(21) Appl. No.: 18/633,843

(22) Filed: Apr. 12, 2024

Related U.S. Application Data
(60) Provisional application No. 63/459,086, filed on Apr. 13, 2023.

(51) Int. Cl. A61M 60/13; A61M 60/216; A61M 60/419; A61M 60/538; A61M 60/818

(52) U.S. Cl. CPC A61M 60/13; A61M 60/216; A61M 60/419; A61M 60/538; A61M 60/818

(57) ABSTRACT

An intravascular blood pump may be provided, that may include a catheter a miniaturized solid state pump section coupled to a distal end of the catheter. The pump section may include an electric motor comprising a stator and a rotor operably coupled to the stator, a rigid drive shaft disposed within the pump section where the drive shaft is operably coupled to the rotor, and an impeller housing in which an expandable impeller is housed, the expandable impeller being coupled to the drive shaft.

### CROSS-REFERENCE TO RELATED APPLICATIONS

The present application claims priority to U.S. Provisional Patent Application No. 63/459,086, filed Apr. 13, 2023, the contents of which are incorporated by reference herein in its entirety.

### TECHNICAL FIELD

The present application is related to blood pumps, such as intravascular pumps with onboard electric motors.

### BACKGROUND

Intravascular blood pumps may be introduced into a patient either surgically or percutaneously and used to deliver blood from one location in the heart or circulatory system to another location in the heart or circulatory system. For example, when deployed in the left heart, an intravascular blood pump may pump blood from the left ventricle of the heart into the aorta. Likewise, when deployed in the right heart, an intravascular blood pump may pump blood from the inferior vena cava into the pulmonary artery. Intravascular blood pumps may be powered by a motor located outside of the patient's body via an elongated drive shaft or by an onboard motor located inside the patient's body. Some intravascular blood pump systems may operate in parallel with the native heart to supplement cardiac output and partially or fully unload components of the heart.

An intravascular blood pump for percutaneous insertion is typically delivered into the patient tethered to a catheter. The catheter may extend along a longitudinal axis from a distal end to a proximal end, with the pumping device being attached to the catheter at the end remote (distal) from an operator, such as a surgeon. The pumping device may be inserted through the femoral artery or the aorta into the left ventricle of a patient's heart by operation of the catheter. The blood pumps are often provided with an atraumatic tip at their far distal end (i.e., distal of the pumping device). The atraumatic tip mitigates any damage to the patient's soft tissue as the blood pump is positioned into the patient's heart.

### BRIEF SUMMARY

In various aspects, an intravascular blood pump may be provided. The blood pump may include a catheter and a pump section coupled to a distal end of the catheter. The pump section may include an electric motor comprising a stator and a rotor operably coupled to the stator. The rotor and stator may be coaxially aligned. The pump section may include a rigid drive shaft disposed within the pump section, the drive shaft operably coupled to the rotor. The pump section may include an impeller housing (which may be an expandable impeller housing) in which an expandable impeller is housed, the expandable impeller being coupled to the drive shaft. In some embodiments, the expandable impeller may be coupled to the drive shaft at a location distal to the rotor. The pump section may include a motor housing disposed around the electric motor, and a portion of the drive shaft. An outer surface of the motor housing may be partially disposed within the catheter. An outer surface of the motor housing may be partially disposed within the impeller housing. An outer diameter at a distal end of the motor housing may be no larger than 12 french, and preferably no larger than 10 french. The pump section may include a housing connector coupling the catheter and the motor housing. The housing connector may, e.g., have a proximal end disposed within the catheter, and a distal end disposed around a proximal end of the motor housing.

The pump section may include at least one bearing. At least one bearing may be positioned within a motor housing disposed around the electric motor. In some embodiments, at least one of those bearing may be a ceramic bearing.

In some embodiments, the drive shaft may be ceramic.

The intravascular blood pump may include one or more wires extending proximally from the pump section. The one or more wires may be coupled to a circuit disposed within the pump section, the circuit configured to control electricity provided to the stator.

The catheter may include a purge fluid lumen extending through the catheter, and the purge fluid lumen may be operably coupled to the pump section. The catheter may be a straight catheter, or may include a defined (e.g., preformed) bend.

The intravascular blood pump may include downstream tubing coupled to the impeller housing and to a portion of the catheter. The downstream tubing may include a slit extending from a proximal end of a blood flow outlet to a proximal end of the downstream tubing. The catheter may include a defined (e.g., preformed) bend, and the bend may occur inside the downstream tubing. A motor housing may be disposed inside the downstream tubing.

The intravascular blood pump may include a flexible atraumatic tip coupled to a distal end of the impeller housing. In some embodiments, the impeller housing may have a compressed state and an expanded state. The impeller housing may have a maximum outer diameter of, e.g., no more than about 10 French (10F) in the compressed state, and may have a maximum outer diameter of, e.g., no more than about 21 French (21F) in the expanded state.

The intravascular blood pump may include a mesh filter at a blood flow inlet.

In various aspects, a system may be provided. The system may include an intravascular blood pump as disclosed herein, and a controller operably coupled to the intravascular blood pump, the controller configured to control a rotational speed of the impeller.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of an exemplary intravascular blood pump positioned within a left ventricle of a heart.

FIG. 2 is an illustration of an exemplary intravascular blood pump.

FIG. 3 is an illustration of a pump section of the exemplary intravascular blood pump of FIG. 2.

FIGS. 4 and 5 are illustrations of exemplary flexible atraumatic tips.

FIGS. 6 and 7 are illustrations of exemplary intravascular blood pumps with a catheter and downstream tubing.

FIG. 8 is an illustration showing a portion of an exemplary pump section.

FIG. 9 is an illustration showing a portion of an exemplary intravascular blood pump including a pump section and downstream tubing.

FIGS. 10 and 11 are illustrations of exemplary blood pumps having an extended cannula, either with a defined bend (10) or without such a bend (11).

FIGS. 12 and 13 are side views of an impeller housing in an expanded (12) and compressed (13) configuration.

FIG. 14 is a side view of a housing, outflow tube, and an expandable filter.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

In some instances, if the pumping function of the patient's heart is still insufficient despite attempted medical treatment, the circulatory system can be assisted by a ventricular assist device (VAD), such as an intravascular blood pump. Intravascular blood pumps are typically introduced through a blood vessel at an access point in an extremity of the patient's body (although other locations may be utilized, such as via a subclavian artery, etc.), and passed through one or more blood vessels until the pump is positioned in a target location. The pump may be configured to collect blood from the blood circulation at the inlet and to eject it back into the blood circulation at the outlet. In doing so, the VAD needs to overcome the pressure differential between the outlet and the inlet, i.e., the pressure differential between the VAD after and before loading. Such pumps can assist or even replace the inadequate ventricular pumping function of the heart by delivering blood in the same way as the ventricles.

In order to completely replace the pumping function of the heart, the pump must be able to provide a sufficient flow rate of blood while also overcoming the pressure differential. While use of a large diameter pump would solve those technical problems, such large pumps introduce numerous challenges. For example, if the inner diameter of a blood vessel is smaller than the outer diameter of the pump, the pump will not be able to pass safely through the blood vessel. Thus, the smaller the pump, the more likely a pump can pass safely through a patient to a target location, but as pump size is reduced, it becomes more challenging to provide the required flow rates and overcome the pressure differentials.

The present disclosure provides devices, systems, and techniques for, e.g., reducing pump diameter while maintaining a target flow rate at a desired differential pressure.

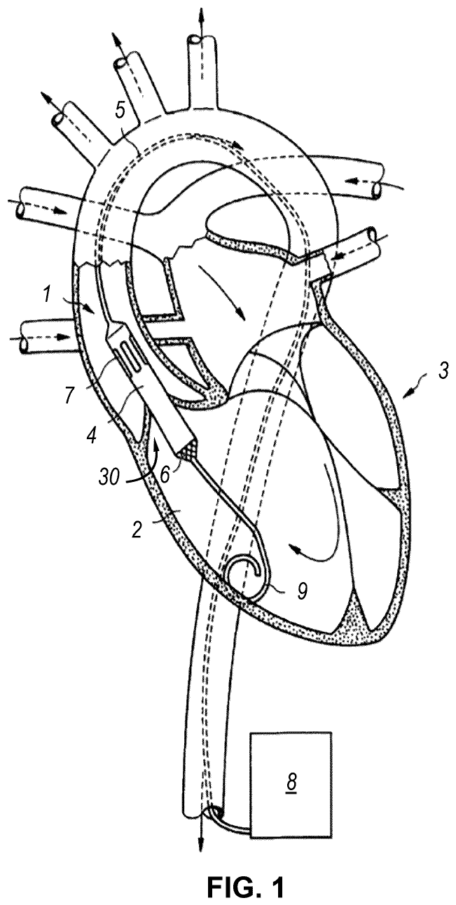

FIG. 1 shows an exemplary use of an intravascular blood pump 1 for supporting a left ventricle 2 of a human heart 3. The intravascular blood pump 1 may include a catheter 5 and a pump section 4 mounted at a distal end region of the catheter 5. The intravascular blood pump 1 may be placed inside the human heart 3 using a percutaneous, transluminal technique. For example, the intravascular blood pump 1 may be introduced through a femoral artery. Likewise, the intravascular blood pump 1 may be introduced through other vessels, such as through the subclavian artery. As shown in FIG. 1, the catheter 5 may be pushed into the aorta such that at least some of pump section 4 reaches through the aortic valve 30 into the heart.

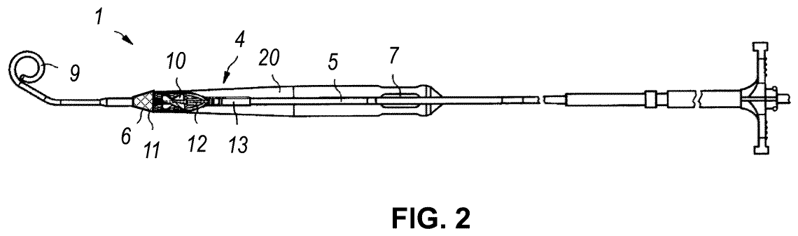

Referring to FIG. 2, an intravascular blood pump 1 can be seen, having a pump section 4 coupled to a distal end of catheter 5. A flexible atraumatic tip 9 may be coupled to a distal end of the pump section 4. The pump section 4 may include an impeller 10 within an impeller housing 11 (which may be an expandable impeller housing), the impeller 10 being operably coupled to an electric motor 13 via a rigid drive shaft 12. In some embodiments, when the impeller rotates, blood is configured to flow through a blood flow inlet 6, through the pump section, into downstream tubing 20, and out a blood flow outlet 7. Impeller 10 may be a compressible and expandable impeller. For example, impeller 10 may be in a compressed state when impeller housing 11 is in a compressed state when the pump section 4 of the intravascular blood pump 1 is introduced into a patient's vasculature.

In some embodiments, the impeller 10 may be coupled to two or more electric motors. In some embodiments, one electric motor may be disposed proximal to the impeller and one electric motor may be disposed distal to the impeller.

In some embodiments, one electric motor 13 may be disposed proximal to the impeller 10 and distal to the blood flow outlet 7.

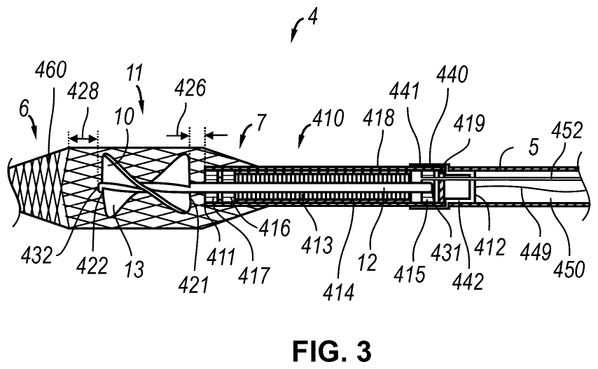

Referring to FIGS. 2 and 3, pump section 4 may include a drive section 410. The drive section may have a distal end 411 and a proximal end 412. Drive section 410 may include an electric motor formed from a stator 414 operably coupled to a rotor 413, configured such that the rotational speed of the rotor can be controlled based on electrical properties of electrical current provided to electromagnets of the stator. The rotor 413 and stator 414 may be coaxially aligned. The stator and rotor may be concentrically positioned. At least a portion of the stator may be disposed around the rotor.

Drive section 410 may include a plurality of bearings, including one or more proximal bearings 415 and one or more distal bearings 416, 417. In some embodiments, at least one bearing is a radial bearing. In some embodiments, at least one bearing is an axial bearing.

Pump section 4 may include a motor housing 418. Motor housing 418 may be disposed around the electric motor (e.g., the rotor and stator). In some embodiments, the motor housing 418 may be disposed around the entire drive section 410. In some embodiments, the motor housing 418 may be disposed around the rotor 413, the stator 414, and at least one bearing (e.g., a proximal bearing 415 and a distal bearing 416). One or more of the bearings may include a ceramic bearing (e.g., be at least partially formed of a ceramic material). In some embodiments, one or more bearings may be a full ceramic bearing.

In some embodiments, the motor housing and the impeller housing form a single combined housing (e.g., the motor housing and impeller housing are formed from a single layer, or otherwise form a continuous single housing).

Motor housing 418 may have an outer diameter at a distal end that is no larger than an outer diameter of catheter 5. Motor housing 418 may have an outer diameter at a distal end that is larger than an outer diameter of catheter 5. Motor housing 418 may have an outer diameter at a distal end that is no larger than 12 French (12F), preferably no larger than 10 French (10F). Motor housing 418 also may have an outer diameter at a distal end that is no larger than 3.3 mm±0.5 mm. Motor housing 418 also may have an outer diameter at a distal end that is no larger than 4.0 mm±0.5 mm.

In some embodiments, the outer diameter of the motor housing may vary. Motor housing 418 may have an average outer diameter that is no larger than an outer diameter of catheter 5. Motor housing 418 may have an average outer diameter that is larger than an outer diameter of catheter 5. Motor housing 418 may have an average outer diameter that is no larger than 12 French (12F), preferably no larger than 10 French (10F). Motor housing 418 also may have an average outer diameter that is no larger than 3.3 mm±0.5 mm. Motor housing 418 also may have an average outer diameter that is no larger than 4.0 mm±0.5 mm.

In some embodiments, motor housing 418 may have a constant outer diameter. In some embodiments, motor housing 418 may have a diameter at the proximal end that is larger than a diameter at a distal end.

Motor housing 418 may be coupled to catheter 5. In some embodiments, a proximal end of motor housing 418 may be disposed within a distal end of catheter 5. In some embodiments, a proximal end of the motor housing 418 may not be disposed within a distal end of the catheter 5. In some embodiments, pump section 4 may include a housing connector 440. The housing connector may be used to couple a motor housing to a catheter. This may include any appropriate means for doing so, including, e.g., axially welding two components together, or bonding two components together. In some embodiments, Housing connector 440 may have a proximal portion 442 disposed within a distal end of catheter 5, and a distal portion 441 disposed around a proximal end of motor housing 418.

The rotor 413 may be coupled to a drive shaft 12. The drive shaft 12 may be coupled to an impeller 10 disposed within an impeller housing 11 (where the impeller housing 11 may form a cage around the impeller 10), the impeller 10 being configured to cause blood to flow from a blood flow inlet 6 at a distal end of the pump section 4 to a blood flow outlet 7 located proximally of the blood flow inlet 6. Note, as used herein, the term “impeller” is used to avoid confusion with the “rotor” 413 of the electric motor. However, the term “impeller” as used herein is intended to refer to any rotating component containing one or more blades 13, where the blades are configured to cause blood to move from the blood flow inlet towards the blood flow outlet when the impeller rotates.

A proximal end 431 of the drive shaft 12 may be disposed within the motor section 410, such as, e.g., within a proximal bearing 415, and the drive shaft 12 may extend distally, extending beyond a distal end 411 of the motor section 410. In some embodiments, the distal end 432 of the drive shaft may extend to the distal end 422 of the impeller 10. In some embodiments, the distal end 432 of the drive shaft may extend distally beyond the distal end 422 of the impeller 10. In some embodiments, there is an axial gap 428 between the distal end 432 of the drive shaft 12 and the distal end of the impeller housing 11. In some embodiments, there is an axial gap between the distal end 432 of the drive shaft 12 and the distal end of the blood flow inlet 6. In some embodiments, the drive shaft 12 does not extend into catheter 5.

In some embodiments, there is an axial gap 426 between the proximal end 421 of the blades of impeller 10 and the distal end 411 of the motor section 410.

The drive shaft 12 may be composed of a rigid material. In some embodiments, drive shaft 12 can be composed of a breakproof ceramic, diamond, a stainless steel (such as 1.4441, 316 L, etc.), a cobalt alloy (such as MP35N, 35N LT, etc.), or a combination thereof. The drive shaft 12 may be coated with an amorphous carbon coating (DLC=diamond-like carbon or diamond-like coating). DLC layers may be especially wear-resistant and low-friction. They may be only a few micrometers thick and can be produced for example by chemical vapor deposition (CVD) or physical vapor deposition (PVD). Impeller 10 may be made from a compressible and expandable plastic material. For example, impeller 10 may be directly casted or glued onto drive shaft 12.

By placing the blood flow inlet 6 inside the left ventricle 2 and the blood flow outlet 7 inside the aorta, the intravascular blood pump 1 may support the patient's systemic blood circulation. If the intravascular blood pump 1 is configured and placed differently, it may be used, e.g., to support the patient's pulmonary or kidney blood circulation instead.

Both the impeller 10 and the impeller housing 11 may be made compressible, such that the intravascular blood pump 1 may be inserted into and/or through the patient's vascular system while both the impeller 10 and the impeller housing 11 are in their compressed state, and such that the impeller 10 and the impeller housing 11 may be expanded once the pump section 4 is positioned at or near its target location in the patient's heart. Impeller housing 11 may thus be an expandable impeller housing 11. In the compressed state, impeller housing 11 may have an outer diameter of about 12 French (12F).

In the compressed state, impeller housing 11 may have any appropriate outer diameter. In the compressed state, impeller housing 11 may have an outer diameter of no more than about 10 French (10F). In the compressed state, impeller housing 11 may have an outer diameter of no more than about 11 French (11F). In the compressed state, impeller housing 11 may have an outer diameter of no more than about 12 French (12F). In the compressed state, impeller housing 11 may have an outer diameter of no more than about 13 French (13F). In the compressed state, impeller housing 11 may have an outer diameter of no more than about 14 French (14F). In the compressed state, impeller housing 11 may have an outer diameter of no more than about 15 French (15F). In the compressed state, impeller housing 11 may have an outer diameter of no more than about 16 French (16F). In the compressed state, impeller housing 11 may have an outer diameter of no more than about 17 French (17F). In the compressed state, impeller housing 11 may have an outer diameter of no more than about 18 French (18F).

In the expanded state, impeller housing 11 may have any appropriate outer diameter. In the expanded state, impeller housing 11 may have an outer diameter of no more than about 21 French (21F). In the expanded state, impeller housing 11 may have an outer diameter of no more than about 22 French (22F). In the expanded state, impeller housing 11 may have an outer diameter of no more than about 23 French (23F). In the expanded state, impeller housing 11 may have an outer diameter of no more than about 24 French (24F). In the expanded state, impeller housing 11 may have an outer diameter of no more than about 25 French (25F). In the expanded state, impeller housing 11 may have an outer diameter of no more than about 26 French (26F). In the expanded state, impeller housing 11 may have an outer diameter of no more than about 27 French (27F). In the expanded state, impeller housing 11 may have an outer diameter of no more than about 28 French (28F). In the expanded state, impeller housing 11 may have an outer diameter of no more than about 29 French (29F). In the expanded state, impeller housing 11 may have an outer diameter of no more than about 30 French (30F).

For example, in some embodiments, expansion may occur when the impeller housing 11 is in the ventricle, the ascending aorta, or the descending aorta. Likewise, in some embodiments, expansion may occur directly after the impeller housing 11 is introduced into the patient's vasculature, with the impeller housing 11 then being moved to its target location in the patient's heart in its expanded state.

As will be appreciated, expansion may occur in any suitable location within the patient's vasculature, such as a portion of the patient's vasculature having a diameter that exceeds the diameter of the expanded impeller housing 11. In some embodiments, the impeller 10 and impeller housing 11 may be formed from any suitable material or materials. For example, in some aspects of the technology, the impeller 10 and/or impeller housing 11 may be composed at least in part from polyurethane, silicone rubber, a shape-memory material such as Nitinol or Ultra-Stiff Nitinol (“USN”), etc.

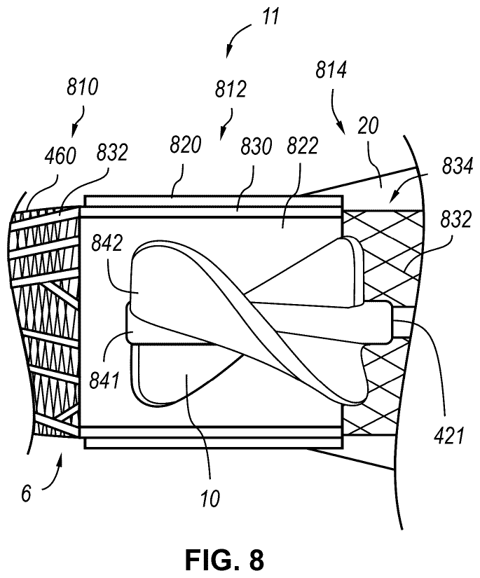

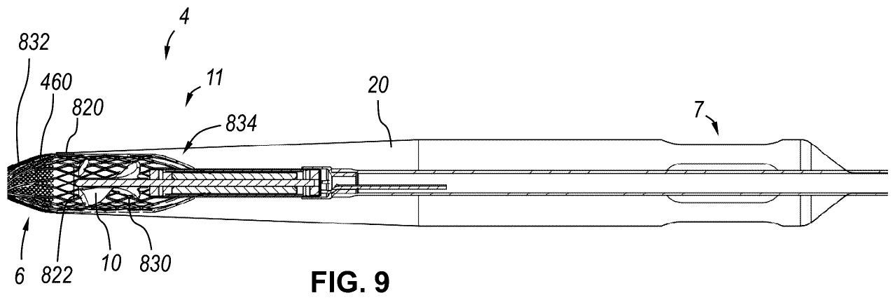

Referring briefly to FIGS. 8 and 9, in some embodiments, the impeller housing 11 may comprise a frame 830 that may include multiple struts 832. The frame may be a shape-memory material, such as a nitinol structure, and may be expandable and compressible. The impeller housing 11 may have different sections. For example, in some embodiments, the impeller housing 11 may have a distal section 810, an intermediate section 812, and a proximal section 814. The distal section 810 may define the blood flow inlet 6, and may include a mesh filter 460. The mesh filter 460 may be operably coupled to one or more struts 832. In some embodiments, the mesh filter may be coupled to an outer lining coating 820 and to an atraumatic tip section if present, but is free of any coupling to the struts 832.

The intermediate section 812 may be disposed around the impeller 10, and may extend in an axial direction at least partially along the length of the impeller 10. That is, in some embodiments, a proximal end 421 of the impeller 10 may extend into proximal section 814. Depending on the design of the impeller, the proximal end may include portions of the hub 841 of the impeller and/or portions of one or more blades 842 that extend into proximal section 814.

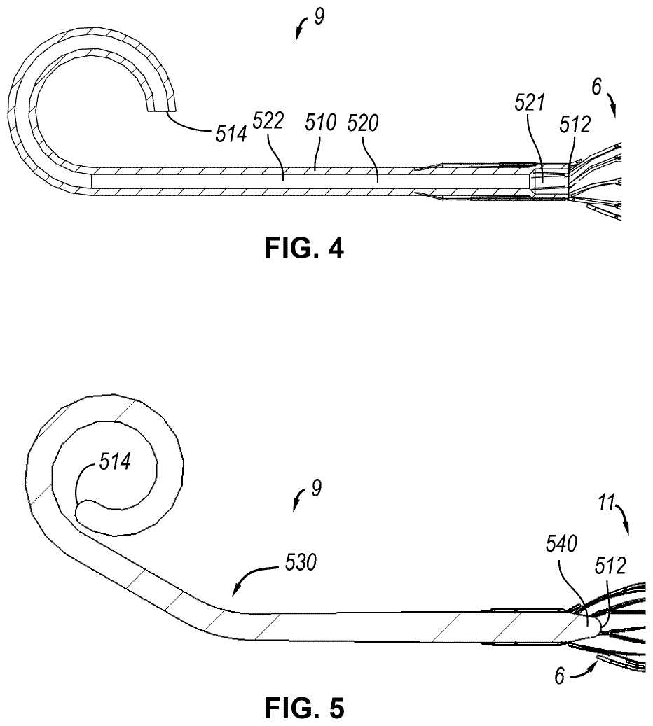

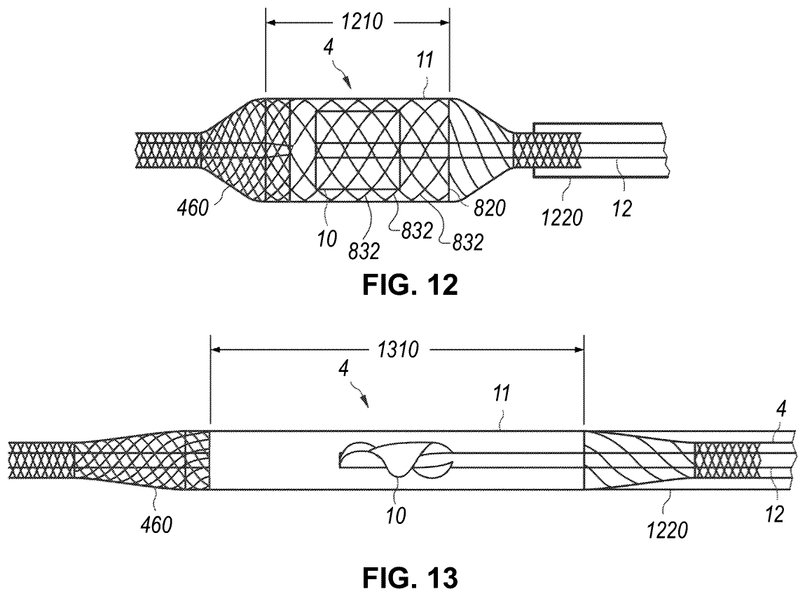

FIGS. 12 and 13 show enlarged side views of an expandable impeller housing (impeller housing 11) of an intravascular blood pump, as well as an expandable filter (mesh filter 460). FIG. 3 shows the expandable housing and the expandable filter in their expanded states, and FIG. 4 shows them in their compressed states. If the impeller housing 11 and the impeller 10 are expandable, the housing may include a plurality of struts, represented by struts 832, made of a suitable shape memory, hyperelastic or superelastic material, such as Nitinol. Hyperelastic materials are typically elastomers. Many such elastomers can elastically deform up to about 100%. Some superelastic materials can elastically deform up to about 6-8%. Nitinol is a trade name for a nickel-titanium alloy distinguished from other materials by its shape memory and superelastic characteristics.

Struts 832 may be made of wire or other filament. As shown in FIGS. 12 and 13, impeller housing 11 provides a cage around the impeller 10. When radially expanded (FIG. 12), the length 1210 of the impeller housing 11 may be less than the length 1310 when the impeller housing 11 is radially compressed (FIG. 4). The change in length 1310 to 1210 may be due to unwinding of the struts 832, when the impeller housing 11 expands. In some embodiments, the change in 1310 to 1210 may be about 1-2 mm.

The expandable housing 11, expandable impeller 10 and expandable filter 460 may be kept in their compressed states by a suitable compression sleeve 1220 slid over the expandable housing 11, expandable impeller 10 and expandable filter 460. The intravascular blood pump, may be transported through the patient's vascular system while the housing, impeller, and filter are in their compressed states. Once the pump section 4 is at its target location, the housing 11, the impeller 10 and the filter 460 may be allowed to expanded, e.g., by pushing the pump section 4 out of the compression sleeve 1220 in a forward (distal) direction or by pulling back (in a proximal direction) the compression sleeve 1220. With the compression sleeve removed, the housing 11 expands, due to its shape-memory, superelastic or hyperelastic properties, as shown in FIG. 3. At the same time, the impeller 10 expands due to its elasticity. As the housing 11 expands radially away from the drive shaft 12, the housing may longitudinally contract to the length 1210.

When the intravascular blood pump is in its expanded state and needs to be removed from the patient, the housing 11 may be pulled back into the compression sleeve 1220, which causes the housing 11 to compress radially, and may cause the housing to longitudinally extend to the length 1310. The filter 460 and the impeller 10 are also compressed. The smaller diameter of the housing thus achieved facilitates removing the intravascular blood pump from the patient through the vasculature. Thus, the housing, the impeller and the filter may each be configured to be alternatingly radially compressed and radially expanded.

The intermediate section 812 may include an outer liner or coating 820 and an inner liner or coating 822 disposed around frame 830. These liners or coatings may be configured to prevent blood from exiting the impeller housing in the intermediate section 812 (e.g., keeping blood moving in a generally axial direction, rather than radially outward out of the impeller housing). The inner and outer liners or coatings may be composed of, e.g., a polyurethane (PU). The proximal section 814 may include one or more openings 834 (such as openings between struts 832) that allow blood to flow out of the pump section and into downstream tubing 20. In some embodiments, downstream tubing 20 may be coupled to the outer liner or coating 820. In some embodiments, downstream tubing 20 may be disposed over outer liner or coating 820. In some embodiments, downstream tubing 20 may be disposed under outer liner or coating 820. In some embodiments, outer liner 820 and downstream tubing 20 may be a single element (e.g,. in some embodiments, the downstream tubing and outer liner form a continuous single layer).

Motor section 410 may include a circuit board 419 which may be connected to one or more wires 449. The wires may pass through a lumen 450 in the catheter to a controller (see, e.g., controller 8 in FIG. 1), where the controller may be configured to control the rotational speed of the motor.

The controller may also control flow of a purge fluid through a purge fluid lumen or line in the catheter and/or control the pressure of the purge fluid, to allow purge fluid to be transported into the pump section 4. In some embodiments, the drive shaft 12 may be hollow along some or all of its length, such that it may also function as a conduit for the purge fluid. In some embodiments, purge fluid may be transported through pump section 4. In some embodiments, purge fluid may be transported through one or more bearings (e.g., bearings 415, 416, and/or 417) of pump section 4.

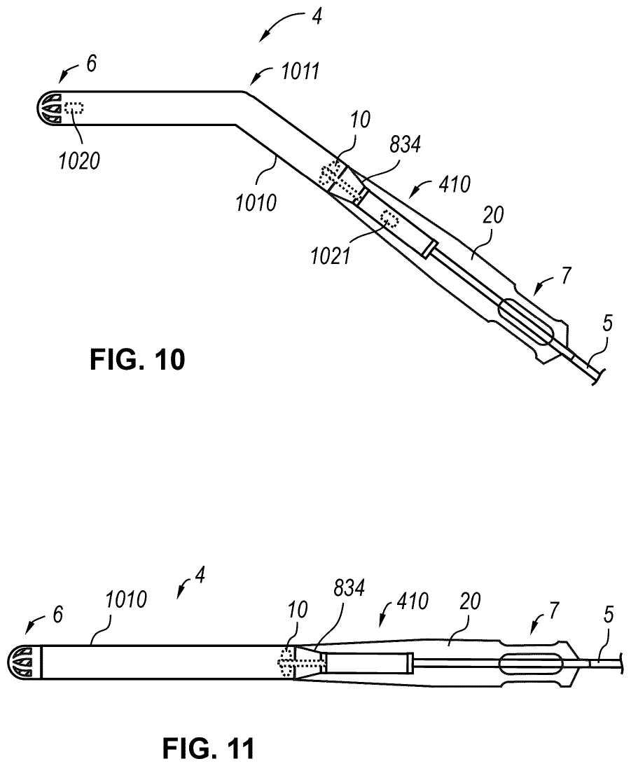

Referring to FIG. 10, in some embodiments, the blood pump may include one or more sensors. In some embodiments, the sensors may be disposed in or on the blood pump. For example, a sensor 1020 may be disposed at a distal end of the blood pump. A sensor 1021 may be disposed on a motor section 410. A sensor (e.g., sensor 1021) may be disposed within the downstream tubing 20, prior to the blood flow outlet 7 but after opening(s) 834 that allow blood to exit the pump section and enter the downstream tubing. In some embodiments, the sensors may include a pressure sensor, which may be an optical pressure sensor. The controller may be configured to control the blood pump based on information received from one or more of the sensor(s) in or on the blood pump.

As shown in FIG. 3, impeller housing 11 may include a mesh filter 460 at the blood flow inlet, e.g., to filter any blood before the blood enters the pump section, and to prevent tissue such as traveculae from getting sucked in and damaged in the pump section. The mesh filter may be composed at least in part from, e.g., polyurethane. The mesh filter may be expandable. Any appropriate mesh-pattern geometric configuration (e.g., the cross-sectional shape of openings through the mesh filter) may be utilized—rectangular mesh, square mesh, diamond mesh, hexagon mesh, circular mesh, etc.

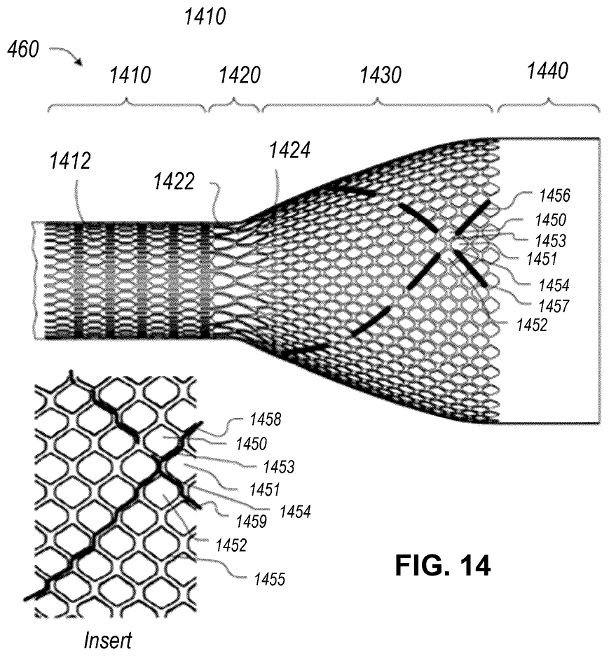

Referring to FIG. 14, in some embodiments, the expandable filter 460 may include a transitional zone 1420 where a distal tubular filter section 1410 and a tapered filter section 1430 meet. Holes, exemplified by hole 1422, in the transitional zone 1420 are longer and wider than adjacent holes of the tapered filter section 1430. Preferably, the holes 1422 in the transitional zone are at least twice as large as the adjacent holes, exemplified by hole 1424, in the tapered filter section 1430. In one embodiment, for each pair of circumferentially adjacent holes 1424 in a row of the tapered filter section 1430, the transitional zone 1420 has one hole 1422 that circumferentially straddles the two holes 1424. Thus, the number of holes in a circumferential row in the transitional zone 1420 may be half the number of holes in a circumferential row in the tapered filter section 1430. In some other embodiments, other ratios may be used, such as 3:1, 4:1 or 3:2. Each hole 1424 in the transitional zone 1420 may be about twice, thrice or another multiple as long (in the longitudinal direction) and about twice, thrice or another multiple as wide (in the circumferential direction) as the hole 1424 in the tapered filter section 1430, depending on the ratio of the number of holes 1424 in one row of the tapered filter section 1430 to the number of holes 1422 in one row of the transitional zone 1420.

The dimensions and shapes of the holes 1450-1452 and 1424 and dimensions of the struts 1456-1457 should be chosen such that, when the tapered filter section 1430 is fully open, the inflow of the housing in its expanded state can be disposed within the tapered filter section 1430, without exceeding limits of clastic deformation of the material. For example, the length of two circumferentially adjacent struts 1456-1457 (on zigzag of a zigzag circumferential ring), multiplied by the number of apertures 1450-1452 in a circumferential row, should about equal the circumference of a fully-expanded housing, taking into account any local elastic deformation of the filter material.

The apertures 1450-1452 are positioned such that material, exemplified by material 1453, 1454, and 1455, between the apertures 1450-1452 forms first and second struts. Two exemplary struts 1456 and 1457 are indicated in FIG. 14 by heavy dashed lines. As noted, a generally helical curve may include minor zigzags, not necessarily all the same, as exemplified by generally helical curves 1456 and 1457. These zigzags are more clearly seen in the insert in FIG. 14, for example in struts 1458 and 1459, which are indicated by heavy solid and dashed lines.

Adjacent holes 1422 in the transitional zone 1420 are separated from each other by struts that are wider than an adjacent strut 1456-1457 of the tapered filter section 1430. These wider struts stabilize the larger holes 1424. In some embodiments, the holes 1422 in the transitional zone 1420 may be chosen to be larger than holes in the tapered filter section 1430.

As can be seen, the holes 1424 in a distal region of the tapered filter section 1430 are narrower, in a circumferential direction, than the holes 1450-1452 in a proximal region of the tapered filter section 1430. In other words, sizes of the apertures 1450-1452 increase monotonically in the proximal direction, along the longitudinal axis. In addition, in the distal tubular filter section 1410, the holes 1412 take the form of narrow axial slits, which are offset from each other in a circumferential direction. This is advantageous, as narrow holes can widen when the expandable filter 460 is expanded at the distal tubular filter section 1410 and the distal region of the tapered filter section 1430, such as when the impeller 10 is inserted into the housing. Wider holes are bounded by thicker struts, particularly in the tapered filter section 1430. The struts have a width of between about 30 μm in the distal region, and about 60 μm in the proximal region, of the tapered filter section 1430. Preferably, the largest diameter of the holes in the tapered filter section 1430 is between about 300 μm and about 500 μm.

As shown in FIGS. 1 and 2, the pump section 4 can be coupled to a flexible atraumatic tip 9 at its distal end. (e.g., The flexible atraumatic tip 9 may have any suitable shape, such as a pigtail or a J-form, and may be configured to facilitate placement of the intravascular blood pump 1 by aiding navigation inside the patient's vascular system. Furthermore, the softness of the flexible atraumatic tip 9 may be configured to allow the pump section 4 to support itself atraumatically against a wall of the left ventricle 2. In some embodiments, as shown in FIG. 4, the flexible atraumatic tip 9 may include a straight configuration. Likewise, in some embodiments, as shown in FIG. 5, the flexible atraumatic tip 9 may include a defined bend configuration (sec defined (e.g., preformed) “bend” 530 in FIG. 5 that is not present in FIG. 4).

In some embodiments, the flexible atraumatic tip 9 may include a tubular body 510. The tubular body 510 may defining a lumen 520 extending from a proximal end 512 towards a distal end 514 of the atraumatic tip, the lumen extending at least partially through the flexible atraumatic tip 9. Referring to FIG. 4, In some embodiments, the lumen may extend the entire length of the flexible atraumatic, from proximal end 512 to distal end 514. Such a configuration may allow a guidewire (not shown) to pass through the lumen, which may allow the pump 1 to use the guidewire for positioning within a subject. The lumen 520 may have a constant inner diameter. The lumen 520 may have different inner diameters at different locations along the length of the lumen. For example, a proximal end portion 521 may have a first inner diameter, while a distal portion 522 may have a second inner diameter. In some embodiments, the first inner diameter may be larger than the second inner diameter. Referring to FIG. 5, in some embodiments, no lumen may be present. In some embodiments, a conical portion 540 at the proximal end of the tubular body 510 may be present, and may extend, e.g., into an internal volume of space defined by the impeller housing 11, such as near the blood flow inlet 6).

As shown in FIGS. 2, 6, and 7, the pump 1 may include downstream tubing 20 through which the catheter 5 is disposed. The downstream tubing 20 may be made of a flexible material or materials such that it may be compressed by the aortic valve as the patient's heart is pumping. Likewise in some embodiments, the tubing 20 may be configured to expand as a result of a blood flow generated by the rotor during rotation. Blood is preferably configured to be pushed by an impeller through the downstream tubing. Blood is preferably not suctioned through any portion of the downstream tubing.

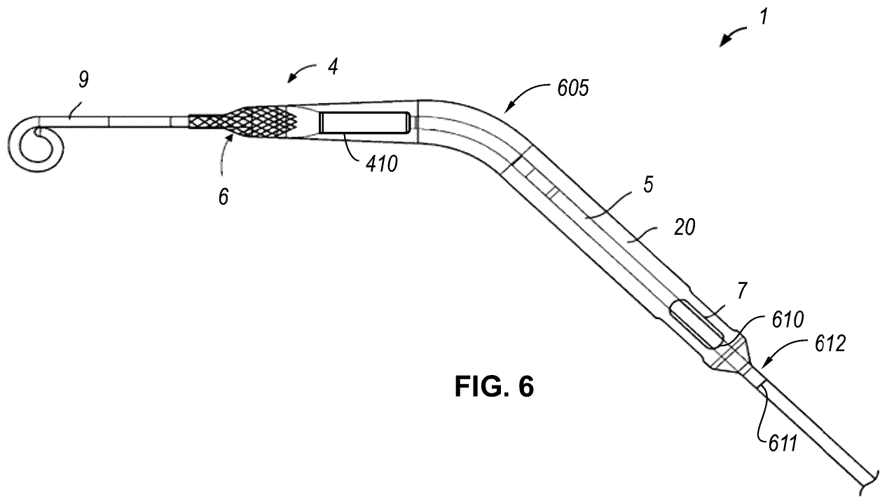

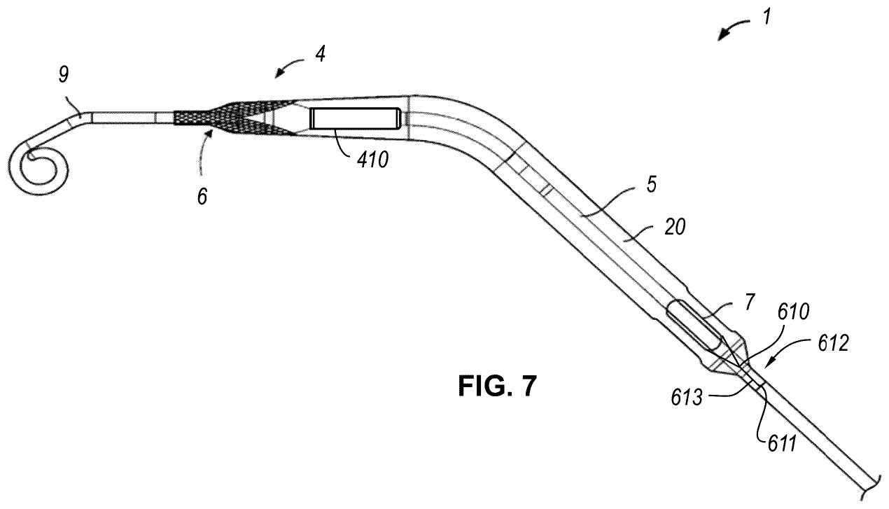

The catheter may preferably be a flexible catheter. The downstream tubing and catheter may have any suitable shape and configuration. For example, as shown in FIG. 2, the downstream tubing 20 and the catheter 5 may include a straight configuration. In other embodiments, as shown in FIGS. 6 and 7, the catheter 5 may include a bent configuration. In such embodiments, the downstream tubing 20 also may include a “defined bend” configuration, with the catheter 5 extending through the bent downstream tubing 20. As will be appreciated, in some embodiments, the catheter 5 also may include one or more straight regions (e.g., downstream or upstream of the defined bend 605), with the downstream tubing 20 also having corresponding straight regions. If a defined bend is present, pump section 4 may be located distal to the defined bend 605.

In other embodiments. The difference in defined bend angles may be used to account for activity of the pump during insertion. For example, to insert the pump in the patient, the pump may first be retracted into an introducer sheath, which is thereafter advanced into the patient's vasculature.

In such embodiments, both the catheter and downstream tubing may remain in a straight configuration in the introducer sheath during delivery. When the pump is thereafter deployed from the introducer and into the patient, the catheter and the downstream tubing may not rebound to the same defined bend angles. For example, in some embodiments, after deployment, the catheter may not return to a 45°±10° defined bend angle. Instead, once deployed from the introducer sheath, the catheter may have a different bend angle. In some embodiments, the initial defined bend angles of the catheter and of the downstream tubing may be configured such that they are different when formed, but will be similar after deployment into the body (and from the introducer sheath).

The length of the downstream tubing 20 between the blood flow inlet 6 and the blood flow outlet 7 may be longer in some embodiments than in others. A longer region of downstream tubing 20 between the blood flow inlet 6 and the blood flow outlet 7 may make it easier to ensure that the pump 1 is placed properly across a valve 30 when the pump is in the patient, and/or that the pump 1 will be less likely to be inadvertently shifted out of its intended position (e.g., shifted such that the blood flow inlet 6 and the blood flow outlet 7 both end up on the same side of the valve 30, shifted such that the blood flow inlet 6 or the blood flow outlet 7 becomes fully or partially covered by valve 30, etc.).

Referring to FIG. 6, in some embodiments, a coupled portion 612 of downstream tubing 20 that is at the proximal end 611 of the downstream tubing may be coupled to an outer surface of catheter 5. The proximal end 610 of the at least one opening forming the blood flow outlet 7 may be located distal to proximal end 611 of the downstream tubing 20, and may be located distal to the coupled portion 612 of the downstream tubing 20, Referring to FIG. 7, in some embodiments, at least one slit 613 may extend from the proximal end 611 of the downstream tubing 20 to the proximal end 610 of the at least one opening forming the blood flow outlet 7. In some embodiments, the slit 613 may connect to the proximal end 610 of the opening forming the blood flow outlet 7 at the distal end of the coupled portion 612. In some embodiments, the distal and/or proximal end(s) 610 of the at least one opening forming the blood flow outlet 7 may be a curved end (e.g., as part of an oval or stadium shaped opening) while in others it may form a pyramidal or triangular-shaped end.

Referring to FIGS. 10 and 11, the pump section 4 may include an extended cannula 1010 operably coupled to the catheter 5. The extended cannula may extend distally from the motor section. The extended cannula may have an axial length that is at least two times an axial length of the impeller. The extended cannula may have an axial length that is at least three times an axial length of the impeller. The extended cannula may have an axial length that is at least five times an axial length of the impeller. The extended cannula may have an axial length that no more than twenty times an axial length of the impeller. The extended cannula may have an axial length that no more than ten times an axial length of the impeller. In some embodiments, the extended cannula may be radially expandable (e.g., having an expanded configuration and a compressed configuration).

In some embodiments, the extended cannula may have a maximum outer diameter no more than 1 mm larger than the average outer diameter of the motor section. In some embodiments, the extended cannula may have a maximum outer diameter that is at least than 1 mm larger than the average outer diameter of the motor section. In some embodiments, the extended cannula may have a maximum outer diameter that is at least than 3 mm larger than the average outer diameter of the motor section. In some embodiments, the extended cannula may have a maximum outer diameter no more than 5 mm larger than the average outer diameter of the motor section. In some embodiments, the extended cannula may have a maximum outer diameter no more than 7 mm larger than the average outer diameter of the motor section. In some embodiments, the extended cannula may have a maximum outer diameter no more than 10 mm larger than the average outer diameter of the motor section.

The extended cannula may be a flexible cannula. The extended cannula may be operably coupled to a distal end of a motor section 410. The extended cannula may be a wire-reinforced cannula (such as a cannula reinforced with nitinol). An impeller 10 may be disposed within the extended cannula. In some embodiments, such as shown in FIG. 10, the extended cannula may include a defined bend 1011. In other embodiments, such as shown in FIG. 11, the extended cannula may be free of such a defined bend.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. An intravascular blood pump, comprising:
a catheter; and
a pump section coupled to a distal end of the catheter, the pump section comprising:
an electric motor comprising a stator and a rotor operably coupled to the stator;
a drive shaft disposed within the pump section, the drive shaft operably coupled to the rotor; and
an impeller housing in which an expandable impeller is housed, the expandable impeller being coupled to the drive shaft.

2. The intravascular blood pump according to claim 1, wherein the expandable impeller is coupled to the drive shaft at a location distal to the rotor.

3. The intravascular blood pump according to claim 1, wherein the pump section further comprises a motor housing disposed around the electric motor, and a portion of the drive shaft.

4. The intravascular blood pump according to claim 3, wherein an outer surface of the motor housing is partially disposed within the catheter.

5. The intravascular blood pump according to claim 3, the motor housing and the impeller housing form a single combined housing.

6. The intravascular blood pump according to claim 3, wherein an outer surface of the motor housing is partially disposed within the impeller housing.

7. The intravascular blood pump according to claim 3, wherein the pump section further comprises a housing connector coupling the motor housing to the catheter.

8. The intravascular blood pump according to claim 3, wherein an average outer diameter of the motor housing is no larger than 12 french, preferably no larger than 10 french.

9. The intravascular blood pump according to claim 1, wherein the rotor and stator of the electric motor are coaxially aligned.

10. The intravascular blood pump according to claim 1, further comprising at least one bearing within a motor housing disposed around the electric motor.

11. The intravascular blood pump according to claim 10, wherein the at least one bearing includes a plurality of bearings, and one or more bearings of the plurality of bearings is a ceramic bearing.

12. The intravascular blood pump according to claim 1, wherein the drive shaft is ceramic.

13. The intravascular blood pump according to claim 1, further comprising one or more wires extending proximally from the pump section.

14. The intravascular blood pump according to claim 13, wherein the one or more wires are coupled to a circuit disposed within the pump section, the circuit configured to control electricity provided to the stator.

15. The intravascular blood pump according to claim 1, further comprising a purge fluid lumen extending through the catheter, the purge fluid lumen being operably coupled to the pump section.

16. The intravascular blood pump according to claim 1, wherein the catheter comprises a defined bend.

17. The intravascular blood pump according to claim 1, further comprising downstream tubing coupled to the impeller housing and to a portion of the catheter, wherein the downstream tubing has one or more blood flow outlets.

18. The intravascular blood pump according to claim 17, wherein the catheter comprises a defined bend, the defined bend occurring inside the downstream tubing.

19. The intravascular blood pump according to claim 17, wherein the downstream tubing is disposed around a motor housing.

20. The intravascular blood pump according to claim 17, wherein the downstream tubing includes a slit extending from a proximal end of a blood flow outlet to a proximal end of the downstream tubing.

21. The intravascular blood pump according to claim 1, further comprising a flexible atraumatic tip coupled to a distal end of the impeller housing.

22. The intravascular blood pump according to claim 1, wherein the impeller housing comprises a mesh filter at a blood flow inlet.

23. The intravascular blood pump according to claim 1, wherein the impeller housing has a compressed state and an expanded state, the impeller housing having a maximum outer diameter of about 10 French (10F) in the compressed state, and a maximum outer diameter of about 21 French (21F) in the expanded state.

24. The intravascular blood pump according to claim 1, wherein the pump section forms an extended cannula extending distally from a motor housing.

25. The intravascular blood pump according to claim 24, wherein the expandable impeller is disposed within the extended cannula.

26. The intravascular blood pump according to claim 24, wherein the extended cannula includes a defined bend distal to the expandable impeller.

27. The intravascular blood pump according to claim 1, where there is an axial gap between a proximal end of blades of the expandable impeller and a distal end of a motor section.

28. A system, comprising:
an intravascular blood pump according to claim 1; and
a controller operably coupled to the intravascular blood pump, the controller configured to control a rotational speed of the electric motor.
